(19) United States
(12) Patent Application Publication
(10) Pub. No.: US 2004/0068284 A1
(43) Pub. Date: Apr. 8, 2004

(54) Method for stimulating hair growth and kit for carrying out said method

(76) Inventor: Thomas H. Barrows, Austell, GA (US)

Correspondence Address: MICHAEL BEST & FRIEDRICH, LLP, ONE SOUTH PINCKNEY STREET, P O BOX 1806, MADISON, WI, 53701

(21) Appl. No.: 10/470,389

(22) PCT Filed: Jan. 29, 2002
(86) PCT No.: PCT/US02/02643

(51) Int. Cl. A61F 2/10; A61B 17/34

(52) U.S. Cl. 606/187; 128/898; 623/15.11

(57) ABSTRACT

Methods and devices are disclosed for use in the treatment of male pattern baldness and other conditions involving hair loss by facilitating the growth of new hair in the dermis of a living subject. The method involves the steps of atraumatically injecting follicle progenitor cells into the interface between the dermis and the epidermis and allowing the cells to grow into new hair follicles.

### CROSS-REFERENCE TO RELATED APPLICATIONS

This application claims the benefit of priority from U.S. Provisional Patent Application Serial No. 60/264,806, filed Jan. 29, 2001.

### STATEMENT REGARDING FEDERALLY SPONSORED RESEARCH OR DEVELOPMENT

Not Applicable

### FIELD OF TIE INVENTION

This invention relates, generally, to methods of curing male pattern baldness and other conditions involving hair loss by facilitating the growth of new hair in the dermis of a living subject. More specifically, the invention relates to a method of growing new hair from a culture of follicle progenitor cells injected into the skin of a living subject.

### BACKGROUND OF THE INVENTION

Male pattern baldness is a common condition that is often treated by hair transplant surgery. In this procedure hair follicles from areas of the scalp that are not within the baldness pattern are excised and re-implanted to create the illusion of a fuller head of hair. In fact, no new hair is created by this procedures which is limited by the number of follicles that can be harvested for re-distribution.

It is well known that specific types of cells found in specific sub-structures within the hair follicle have the capacity to induce the formation of complete, normally functioning hair follicles. Such cells are known as follicular stem cells or follicle progenitor cells. See the following articles, the teachings of which are incorporated herein, for illustrations of experimental work illustrating the function of various types of follicular stem cells or progenitor cells in new hair formation in vitro and in the dermal tissue of various organisms: R. M. Lavker et al., Journal of Investigative Dermatology, 101(1), Supplement, July 1993, 16S-26S; W. C. Weinberg, et al., Journal of Investigative Dermatology, 100(3), March 1993, 229-235; J. Kamimura, et al., Journal of Investigative Dermatology, 109(4), October 1997, 534-540; Lichti, A. B, et al., Journal of investigative Dermatology, 104(5), Supplement, May 1995, 43S-44S; S. H. Yuspa, et al., Journal of Investigative Dermatology, 101(1), Supplement, July 1993, 27S-32S; and C. A. B. Jahoda, et al., Journal of Investigative Dermatology, 101(1), Supplement, July 1993, 33S-38S.

Previous attempts to exploit this knowledge, for example by injecting or implanting cultured dermal papilla cells into human skin as disclosed in International Publication Number WO 99/01034, the teachings of which are incorporated herein, have generally not been successful. Thus there remains a need, therefor, for methods of creating new hair follicles in the skin of humans, e.g., the scalp, that do not involve painful procedures such as the transplantation of skin or hair.

### BRIEF SUMMARY OF THE INVENTION

In one aspect, the present invention is a method of creating new hair in the skin of a living subject, the skin comprising an epidermis and a dermis layer. The method involves the steps of injecting follicle progenitor cells into the skin and allowing the cells to grow into new hair follicles. Preferably, the follicle progenitor cells are delivered into the skin in the form of a cluster of cells. It is also preferred that the cluster of cells be delivered to the skin atraumatically, preferably to the interface between the dermis and the epidermis. Traumatic methods of implanting cells such as by surgical incision or suction blistering generally have been found to interfere with the survival and/or proper functioning of the implanted cells and typically do not result in follicle induction and hair growth.

In another aspect, the present invention is a method of creating new hair in the skin of a living subject comprising the steps of isolating follicle progenitor cells, growing the cells in culture, creating clusters of cells, and injecting the clusters of cells into the skin. As is noted above, the cell clusters preferably are atraumatically delivered, preferably to the epidermis/dermis interface of the skin.

In yet another aspect, the present invention is a method of atraumatically implanting cells, particularly clusters of cultured follicle progenitor cells, into a space resulting from the temporary separation of the epidermis from the dermis by injection of a fluid into the interface between the epidermis and the dermis or pocket substantially without injury to either the dermis or the epidermis, thereby creating a raised bleb, and injecting said cluster of cells into this fluid-filled space. One skilled in the art will appreciate that cells or cell clusters may be implanted into the skin concurrently with the creation of the bleb. The progenitor cells/cell clusters also may be delivered to the bleb subsequent to the formation of the bleb dermis/epidermis interface.

The method of the present invention enables one to create a multitude of new follicles from each follicle that is removed from a patient or from another source of hair follicles, such as another individual. More specifically, the method of the present invention provides a means for curing male pattern baldness and other conditions involving hair loss.

In another aspect, the present invention is a device that serves the purpose both of providing a convenient housing for culturing said cells into a sufficiently large clump and as a tool for injecting said clump of cells into the skin.

Other advantages and a fuller appreciation of the specific attributes of this invention will be gained upon an examination of the following detailed description of preferred embodiments including the attached drawings, and appended claims.

### DESCRIPTION OF THE DRAWINGS

FIG. 1 is a cross-sectional sketch of the epidermis (1) and dermis (2). In view A, a bleb (3) has been created between epidermis (1) and dermis (2) by injection of e.g., a sodium hylauronate solution, by means of a hypodermic needle and syringe (4). In view B, the bleb has been punctured with a sharp instrument e.g. a scalpel, (5) and a clump of cells (6) mounted on the end of a wire (7) has been injected into the bleb through the cut opening (8). Wire (7) has a knot (9) which helps to deposit cell clump (6) in bleb (3). In view C, wire (7) has been partially pulled out of the incision (8) so that the knot (9) in the wire is outside of the incision (8). The wire optionally can be left in the wound for several days as a means of directing epidermal growth toward the injected cells.

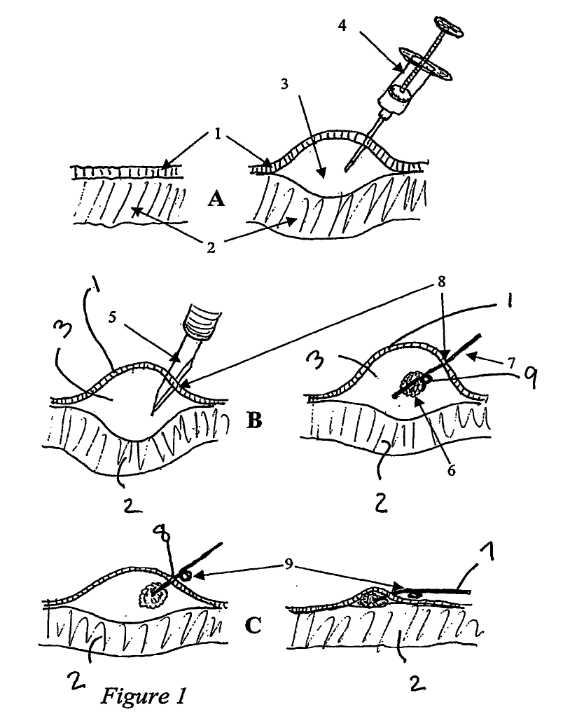

FIG. 2 is a photomicrograph with original magnification of 14× of a structure-forming excipient (10) made from a crosslinked mixture of hyaluronic acid and gelatin on the end of a 0.0035 inch diameter 316-stainless steel wire (11).

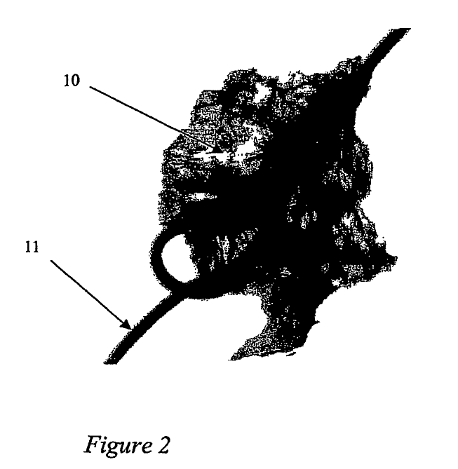

FIG. 3 is a photomicrograph with original magnification of 14× of a structure-forming excipient (12) mounted on the end of a 0.0035 inch diameter wire (11) in which cultured human dermal papilla cells have been seeded onto the excipient and have been growing in culture for 10 days, creating a clump that is a mixture of cells and partially degraded excipient (12).

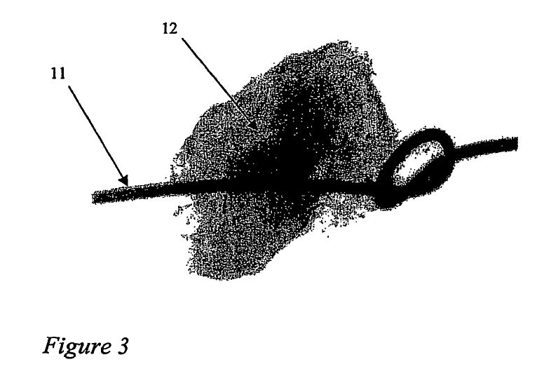

FIG. 4 is a sketch of a device for cell clump injection in which the cell clump/excipient combination (12) is mounted on the end of a wire (11) and packed into the end of a hypodermic syringe needle (13). This needle has a short, intra-dermal bevel (14) with an extra sharp point (15). After penetration of the bleb with this device, the needle (13) is withdrawn while pushing on the wire (11). A knot (16) in the wire (11) prevents the cell clump from riding back on the wire and ensures that it is deposited into the bleb.

### DEFINITIONS

The term “injection”, as used herein, is defined as any procedure utilizing any device by any means to break, cut, breach, puncture or otherwise open the surface of living human skin to deposit a substance into or beneath the skin. The steps of breaking said skin and depositing said substance may be accomplished with the same device or with different devices and may be performed simultaneously, separately in rapid sequence, or after an interval of time between the two steps.

The term “cells” or “injection of cells” or “aliquot of cells”, as used herein, is any of the following:

One or more clusters or clumps of cells of one or more types substantially formed by the adhesion of individual cells to each other with or without the addition of a structure-forming excipient (q.v.).

Clusters or clumps of cells substantially in spherical or bead-like shapes.

Clusters of cells in which two or more different types of cells are organized in each cluster to provide a structure of two or more parts, each part being comprised of primarily one type of cells.

The term “progenitor cell”, as used herein, is defined as any type of cell that has the capacity to transform into a more highly specialized cell and/or recruit and transform surrounding cells into a specialized tissue. Thus cells of the dermal papilla are considered to be progenitor cells because under the proper conditions they induce the formation of hair follicles where none existed previously.

The term “hair follicle neogenesis” or “follicle neogenesis”, as used herein, is defined as the creation of a new, functional, hair follicle in the skin where no functional hair follicle existed previously.

The term “structure-forming excipient”, as used herein, is defined as any non-toxic, tissue compatible, pharmacologically acceptable, bioabsorbable substance that is substantially liquid prior to mixing with, or being added to, living cells and becomes gelatinous, fibrous or substantially non-liquid after being added or mixed with living cells. Structure-forming excipient also means any substance that is solid, gelatinous, fibrous or substantially non-liquid before adding living cells and which liquefies or becomes bioabsorbable thereafter. The function of a structure-forming excipient generally is to facilitate the formation of clusters or clumps of cells by supporting, encapsulating, immobilizing or otherwise causing or facilitating the aggregation or growth resulting in aggregation or clustering of said cells.

The term “bleb”, as used herein, is defined as a fluid filled space, pocket, cavity, cell, or vesicle between the epidermis and the dermis of the skin created by injection of a non-toxic, body-compatible fluid. Creation of a bleb, as the term is intended herein, is a benign, temporary condition that causes substantially no permanent damage to the overlying epidermis or underlying dermis i.e., it is atraumatic.

### DETAILED DESCRIPTION OF THE INVENTION

The present invention comprises a method of inducing the development of new hair follicles that will grow normal, cosmetically useful hair by injecting follicle progenitor cells into a bleb in the skin where the growth of new hair is desired. Cells that possess this follicle-inducing capacity can simply be injected into a bleb in the skin e.g., with a hypodermic needle, as a suspension of cells combined with a structure-forming excipient, or as pre-formed clumps or agglomerates of cells without a structure-forming excipient, or in combination with a structure-forming excipient that has substantially dissolved or degraded during a period of time while said cells were being cultured in vitro.

In a preferred embodiment, the present invention comprises a hair follicle neogenesis method comprising the steps of:

a) providing follicle progenitor cells from biopsied hair follicles;

b) culturing the progenitor cells to increase their number;

c) forming the cultured cells into cell clusters;

d) creating a bleb at the interface of the dermis and epidermis of the skin at a site where one or more new hair follicles are desired; and

e) injecting an aliquot of the cell clusters into the bleb.

The follicle progenitor cells provided in step (a), above, are preferably obtained from the biopsied hair follicles of a live human subject. The patient supplying the biopsy of hair follicles is preferably the same person who receives the injections of cells in step (e). However, it is possible that follicles could be obtained from organ donors or other individuals, whether dead or alive. This would be feasible since follicle progenitor cells are known to be “immune privileged” and are not normally rejected as foreign tissue. Use of organ donor follicles would be especially desired in certain hair-loss conditions where none of the patient's remaining follicles is suitable for biopsy or if the patient is not concerned about duplicating the quality or color of his or her existing hair.

Progenitor cells, suitable for use in the method of the present invention, are located in hair follicle structures such as the dermal papilla, the dermal sheath, and the bulge area It is contemplated that other cells, not generally considered to be progenitor cells, also could be harvested from the biopsy specimen, cultured, and injected into the human subject with the culture of progenitor cells. For example, it may be useful to include epidermal stem cells to facilitate a more rapid induction of follicle neogenesis. Improvements in the methods of culturing progenitor cells useful in the present invention are anticipated. The capacity of cells to induce hair follicle neogenesis also may be improved with the use of growth factors, conditioned media, genetically engineered cells, and the addition of various adjuvants and active agents.

Regarding step (b), above, it is generally believed that repeated culturing of follicle progenitor cells can lead to loss of follicle induction capacity. Thus there may be a limit to the number of cells that can be obtained from the culture of the structure dissected from each individual follicle. However, for a severely bald person it would be possible to obtain subsequent biopsies of follicles from hair that was regenerated by the method of this invention to continue the hair restoration process in stages until the desired result is achieved. Thus, progenitor cells which were themselves progenitor cells may be used in steps (b) and (a). Moreover, it is anticipated that techniques can be developed to obviate limitations in the hair follicle induction capacity of cultured cells, for example by the use of specially developed conditioned media during repeated passaging of the cells. Such improvements are contemplated by the present invention.

Step (c) may be accomplished in a number of ways. For example cell clusters may be formed from substantially individual cell suspensions by (1) encapsulation; (2) adding to the cells a structure-forming excipient, (3) culturing the cells for a period of time in vitro in the presence of a structure-forming excipient such that the cells become adherent to each other and that the excipient is substantially dissolved and replaced with extra-cellular matrix produced by the cells

Referencing step (d), a preferred means for creating the bleb, prior to cell injection, is first to warm the skin with a hot compress to weaken the reversible bond between the epidermis and dernis, then to inject 1% (weight per volume) of e.g., hyaluronic acid, sodium salt, in phosphate buffered saline solution via a fine gauge needle into the skin with the needle tip minimally penetrating the skin. The advantage of the hyaluronic acid is that its viscosity protects the delicate undersurface of the epidermis from subsequent mechanical trauma, and its high molecular weight delays the fluid resorption process. Other high viscosity materials such as polyethylene glycol, chondroitin sulfate, dermatan sulfate, and other polysaccharides, mucopolysaccharides, proteins, glycoproteins and similar polymers, natural and synthetic, may be substituted for hyaluronic acid.

Regarding step (e), above, the number of clumps of cells or cell clusters injected, the size of each cluster, and the volume and composition of the injection fluid may need to be optimized to achieve the best results. The aliquot of cells injected in step (e) of the method may be combined with a fluid that contains a structure-forming excipient, such that the fluid is transformed into a solid at the injection site. This may serve to protect most of the implanted cells from damage caused by post-traumatic inflammation. With the cells temporarily surrounded by an artificial matrix, the inflammation will be confined to the surface of the implant. Thus the more centrally located cells will be protected from this transitory destructive tissue reaction. The structure-forming excipient is preferably a pharmaceutically acceptable carrier. The structure-forming excipient is also preferably bioabsorbable in its solid form, such that once injected into a human host, the structure formed at the injection site is absorbed over time.

An example of one such structure-forming excipient is injectable collagen (Zyderm™, Collagen Aesthetics, Inc.). This product has been combined with cultured human fibroblasts and injected in athymic mice successfully to induce the formation of a viable space-filling implant. Cultured fibroblasts alone survived subcutaneous injection and were accepted as primary takes, but underwent central nodule necrosis when not combined with the collagen excipient. The collagen matrix may have provided an interstitium that was conducive to cell functioning and survival in vivo. These results were published in an article entitled, “Use of Injectable Cultured Human Fibroblasts for Percutaneous Tissue Implantation. An experimental study”, by Remmler D, Thomas J R, Mazoujian G, Pentland A, Schechtman K, Favors S, and Bauer E, in Arch Otolaryngol Head Neck Surg 1989 July;115:837-44, the teachings of which are incorporated herein.

Other structure-forming excipients include Pluronic™ surfactants. These are poly(ethylene oxide-co-propylene oxide) water soluble polymers that can be produced to have a critical solution temperature that coincides with body temperature. Thus cell clumps could be suspended in a cold solution of Pluronic and then injected. The injected liquid would then warm up and become a hydrogel, thereby stabilizing the cell clumps into a matrix to help them survive the initial trauma of implantation.

Other thermally-reversible hydrogels are well known such as those based on N-dimethylisopropylacrylamide. These polymers could be chemically modified to bioabsorb with an appropriate degradation rate.

Other excipients capable of forming structure in situ post in vivo-injection include various two-part cross-linkable liquid systems. In this case the injection device would require two syringes connected to a double-barreled hypodermic needle. The cell clumps would be suspended in one component, part A, (e.g. the one containing a crosslinkable bioabsorbable polymer) and the other component, part B, would contain the crosslinking agent. Polymers and crosslinkers can be chosen from a variety of materials that are biocompatible and bioabsorbable. For example, part A could be fibrinogen and part B could be thrombin. Another example of a pair of ingredients that would be useful in this embodiment of the invention is human serum albumin and poly(ethylene glycol)-disuccinimidyl succinate.

Yet another option for the use of a structure-forming excipient is in the initial in vitro culturing of cells to produce cell clumps with defined size and shape. In this situation it is desirable to have a solid, highly porous excipient to provide a high surface area for cell attachment. As the cells grow, multiply and attach to each other via the production of their natural extra-cellular matrix, the structured excipient is substantially bioabsorbed and replaced with extra-cellular matrix material.

For reasons set forth above, any excipient used in the method of the present invention is preferably selected from the group consisting of: collagen, thermally-reversible hydrogels, chemically crosslinked bioabsorbable polymers, in situ crosslinkable hydrogels, fibrinogen, thrombin, dextrin, amylose, hyaluronic acid, gelatin, chondroitin sulfate, dermatan sulfate, polysaccharides, mucopolysaccharides, proteins, glycoproteins, and any derivative, copolymer, or other modification of the above.

A further option regarding materials implanted along with the injected cells relates to methods for controlling the angle of hair shaft egress from the skin. It is well known that the angle of hair varies on different regions of the scalp and that the uniformity and controlled variation of these angles is important to the cosmetic appearance of a full head of hair. Thus the present invention anticipates improvements in cell implantation methods that facilitate control over the direction and angulation of new hair shafts emerging from the skin due to follicle neogenesis.

One such approach, illustrated in Example 2, is to leave a fine stainless-steel wire in the skin for several days, which causes epidermal down-growth. This growth not only ensures epidermal cell interaction with the implanted follicle progenitor cells, but also provides a tract to the surface of the skin that can serve as a guide for follicle orientation and hair shaft angle control. Other such retained fibers made of synthetic or natural bioabsorbable polymers also are anticipated to be especially useful in this regard because removal would not be required.

The present invention is further illustrated in the following examples, which should not be construed as limiting the scope of the present invention.

### EXAMPLE 1

### Preparation of an Injection Device.

A 4 mm diameter, full-thickness skin punch biopsy is taken from the hair-bearing scalp on the back of the head of a male-pattern baldness patient. The follicle bulbs are cut off from the follicles and the dermal papillae are dissected out. The dermal papilla cells (DP cells) are expanded in culture according to the methods described by A. G. Messenger, British Journal of Dermatology, 110, 685-689 (1984), the teachings of which are incorporated herein by reference.

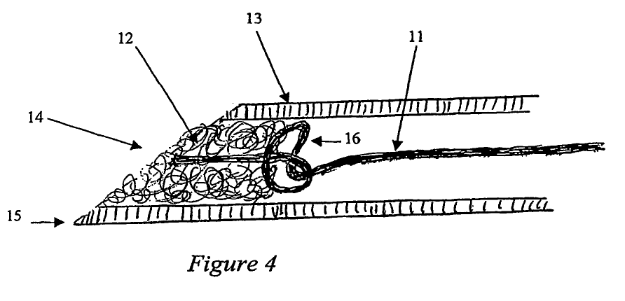

A device of the present invention as illustrated in FIG. 4 is prepared as follows: One hundred milligrams of sodium hyaluronate and 100 milligrams of porcine skin gelatin are dissolved in distilled water to make 10 milliliters of solution (HA-gelatin solution). Particles of low molecular weight poly(d,l-lactide-co-50%-glycolide) (PLGA) are ground and sieved to a particle size range of 100 to 200 microns and mixed with the HA-gelatin solution to form a thick paste. The paste is packed into the tip of an 18-gauge hypodermic needle with a 30-degree point containing a knotted filament of 0.0035-inch diameter stainless steel wire and allowed to dry completely. The PLGA particles are dissolved out with dichloromethane and the residual HA-gelatin excipient crosslinked by soaking in a 0.1% (w/v) solution of 1-ethyl-3-(3-dimethylaminopropyl)carbodiimide (EDC) in a 90:10 (v/v) mixture of acetone and water, respectively. The device is then rinsed with acetone and sterilized by soaking in 70% isopropanol/water and rinsed with sterile water. The cultured DP cells are added to the device by scraping the confluent layer of cells off of the culture dish using the exposed HA-gelatin excipient on the needle. The cell-seeded needle is then placed in a flask of culture media and the cells transferred to the device are allowed to grow and multiply for about one week, during which time the cells become attached to each other as the HA-gelatin excipient is substantially degraded and dissolved.

EXAMPLE 2

### Injection of Cultured Cells.

A patch of bald scalp is first softened with warm water and anesthetized by applying Emla™ lidocaine cream (Astra Pharmaceutical Products, Inc., Westborough, Mass. 01581) and covering with Tegaderm™ dressing (3M, St. Paul, Minn. 55144) for about 30 minutes. The scalp is then wiped clean and then swabbed with 70% isopropanol. A solution of sodium hyaluronate (Healon™, Pharmacia-Upjohn, Kalamazoo, Mich. 49001) is injected into the skin where hair growth is desired after first warming the skin with a hot compress. The resultant bleb is then punctured with the DP cell clump-containing needle described above in Example 1. The needle is withdrawn while holding the fine wire to ensure that the injected cell mass is not withdrawn with the needle. The fine wire is then withdrawn until the knot is outside of the puncture wound. The wire can be removed to complete the procedure or, optionally, it can be left in the wound by taping it to the skin with Tegaderm™ dressing and removed about 5 days later to provide epidermal growth toward the implanted cells. Multiple blebs can be created on the scalp and multiple injections of cells can be made into each bleb to achieve the desired density of new hair.

In about eight weeks post injection, neogenesis of hair follicles is complete and the patient can begin to experience the growth of new, tissue-engineered hair that is perfectly normal and identical to the donor site hair.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation that lawfully can be accorded to the appended claims.

1. A method of creating new hair follicles in the skin of a human subject, the skin comprising dermal and epidermal layers, the method comprising the steps of:
injecting, atraumatically, an aliquot of cultured follicle progenitor cell clusters into a vesicle defined by the dermal and epidermal layers of the skin of the subject and,
permitting the cells to grow to produce new hair follicles.

2. A method of creating new hair follicles in a human subject, comprising:
a) providing follicle progenitor cells derived from biopsied hair follicles;
b) culturing the progenitor cells by allowing them to increase their number;
c) forming the cultured cells into cell clusters;
d) creating a bleb at the interface between the dermis and epidermis of the skin at a site where one or more new hair follicles are desired;
e) injecting an aliquot of the cell clusters into the bleb; and
f) permitting the cells to grow to produce new hair follicles.

3. A method of claim 2 in which the progenitor cells are provided in step a) are obtained from the human subject injected in step (e).

4. The method of claim 2 in which the culture of follicle progenitor cells injected into the skin of the human subject further comprises at least one additional type of cell.

5. The method of claim 4 in which the additional type of cell is obtained from the epidermis.

6. The method of claim 2 in which the culture of follicle progenitor cells are combined with at least one structure-forming excipient prior to being injected into the skin of the human subject.

7. The method of claim 6, wherein the structure-forming excipient is selected from the group consisting of collagen, thermally-reversible hydrogels, chemically crosslinkable polymers, in situ crosslinkable hydrogels, fibrinogen, thrombin, dextrin, amylose, hyaluronic acid, gelatin, chondroitin sulfate, dermatan sulfate, polysaccharides, mucopolysaccharides, proteins, glycoproteins, and any derivative, copolymer, or other modification of these and other pharmaceutically acceptable excipients.

8. The method of claim 6, wherein the structure-forming excipient is a crosslinked mixture of hyaluronic acid and gelatin.

9. The method of claim 2, where the bleb is formed by intra-dermal injection of a viscous solution prepared with the use of solutes selected from the group consisting of hyaluronic acid, polyethylene glycol, chondroitin sulfate, dermatan sulfate, and other polysaccharides, mucopolysaccharides, proteins, glycoproteins and similar polymers, natural and synthetic.

10. The method of claim 2, where the bleb is formed by intra-dermal injection of a solution of sodium hyaluronate.

11. A method according to claim 2 in which injecting step (e) is performed atraumatically.

12. A device for the injection of cultured follicle progenitor cells comprising a hypodermic needle, a plug of cultured follicle progenitor cells contained in the lumen at the tip of the hypodermic needle, and a means for ejecting said plug of cells after injecting said needle into the skin.

13. A device of claim 11 in which the plug of cultured follicle progenitor cells is a mixture of cells and a structure-forming excipient.

14. A device of claim 12 in which the cells are cultured dermal papilla cells and the structure-forming excipient is a crosslinked mixture of hyaluronic acid and gelatin.

15. A method of creating new hair follicles in a human subject, comprising:
a) providing follicle progenitor cells derived from biopsied hair follicles;
b) culturing the progenitor cells by allowing them to increase their number;
c) forming the cultured cells into cell clusters;
d) combining the cell clusters with a structure-forming excipient;
e) creating a bleb, atraumatically, at the interface between the dermis and epidermis of the skin adjacent a site where one or more new hair follicles are desired;
f) injecting an aliquot of the cell clusters into the bleb; and
g) permitting the cells to grow to produce new hair follicles.

16. A method of claim 15 in which the progenitor cells are provided in step (a) are obtained from the human subject injected in step (f).

17. The method of claim 15 in which the culture of follicle progenitor cells injected into the skin of the human subject further comprises at least one additional type of cell.

18. The method of claim 17 in which the additional type of cell is obtained from the epidermis.

19. A method according to claim 15 wherein the structure-forming excipient comprises a crosslinked mixture of hyaluronic acid and gelatin.

20. A method according to claim 15 wherein steps e) and f) are performed concurrently.
